(19) United States
(12) Patent Application Publication
(10) Pub. No.: US 2023/0381163 A1
(43) Pub. Date: Nov. 30, 2023

(54) DABIGATRAN COMPOSITIONS AND METHODS OF TREATING AGE-RELATED MACULAR DEGENERATION

(71) Applicants: The United States Government As Represented By The Department Of Veterans Affairs, Washington, DC (US); MUSC Foundation for Research Development, Charleston, SC (US)

(72) Inventors: Baerbel Rohrer, Charleston, SC (US); Tanjina Akter, Charleston, SC (US)

(21) Appl. No.: 18/323,382

(22) Filed: May 24, 2023

Related U.S. Application Data
(60) Provisional application No. 63/345,253, filed on May 24, 2022.

(51) Int. Cl. A61K 31/4439; A61K 45/06

(52) U.S. Cl. CPC A61K 31/4439; A61K 45/06

(57) ABSTRACT

The disclosure relates to dabigatran and methods of treating age-related macular degeneration in a subject. The method comprises administering to a patient in need of treatment an effective amount of dabigatran.

### CROSS REFERENCE TO RELATED APPLICATIONS

This application claims the benefit of U.S. Provisional Application No. 63/345,253, filed May 24, 2022. The content of this earlier filed application is hereby incorporated by reference herein in its entirety.

### GOVERNMENT SUPPORT

This invention was made with government support under Grant Number EY030072 awarded by the National Institutes of Health and Grant Numbers IK6BX004858, RX000444 and BX002050 awarded by the Department of Veterans Affairs. The government has certain rights in this invention.

### BACKGROUND

Age-related macular degeneration (AMD), the leading cause of irreversible blindness in elderly Caucasian populations, includes destruction of the blood-retinal barrier (BRB) generated by the retinal pigment epithelium-bruch's membrane complex (RPE/BrM), and activation and deposition of complement. Currently therapeutics target and fail to block or cure the disease. Thus, new approaches are needed for treating AMD.

### SUMMARY

Disclosed herein are methods of treating age-related macular degeneration in a subject, the methods comprising: administering to the subject in need thereof a therapeutically effective amount of a direct thrombin-inhibitor.

Disclosed herein are methods of treating age-related macular degeneration in a subject, the methods comprising: (a) identifying a subject in need of treatment; and (b) administering to the subject a therapeutically effective amount of a direct thrombin-inhibitor.

Disclosed herein are methods of inhibiting expression of connective tissue growth factor in a subject, the methods comprising: administering to the subject in need thereof a therapeutically effective amount of a direct thrombin-inhibitor.

Disclosed herein are methods of inhibiting expression of connective tissue growth factor in a subject, the methods comprising: (a) identifying a subject in need of treatment; and (b) administering to the subject a therapeutically effective amount of a direct thrombin-inhibitor.

Disclosed herein are methods of ameliorating one or more symptoms of age-related macular degeneration in a subject, the methods comprising: administering to the subject in need thereof a therapeutically effective amount of a direct thrombin-inhibitor.

Disclosed herein are methods of ameliorating one or more symptoms of age-related macular degeneration in a subject, the methods comprising: (a) identifying a subject in need of treatment; and (b) administering to the subject a therapeutically effective amount of a direct thrombin-inhibitor.

Disclosed herein are methods of reducing complement activation, C3d deposits and/or membrane attack complex of complement (MAC) deposits in a cell, a tissue, a membrane or an extracellular matrix, the methods comprising: contacting a cell or a tissue or administering to a subject in need thereof, a therapeutically effective amount of a direct thrombin-inhibitor.

Other features and advantages of the present compositions and methods are illustrated in the description below, the drawings, and the claims.

### BRIEF DESCRIPTION OF THE DRAWINGS

FIGS. 1A-1C show that dabigatran does not reduce CNV lesion size but does accelerate repair. Argon laser photocoagulation of Bruch's membrane was used to generate four laser spots in each eye of the C57BL/6J mice. Mice were analyzed by OCT on day 5 post laser (FIG. 1A) or on days 5, 9, 12, 15 and 23 during the repair paradigm (FIG. 1B). Mice were fed standard or dabigatran chow (˜10 mg/kg bodyweight per day) during the induction phase of the CNV lesion or during the repair phase, starting at day 5. Quantification of the lesion areas using image J analysis on OCT images demonstrate no reduction in CNV size in the short-term paradigm, and an acceleration of repair in dabigatran-fed mice. Data shown are average values (±SEM) per lesion. FIG. 1C shows the corresponding representative images of CNV lesions are shown.

FIG. 2 shows the thrombin-mediated effects of transepithelial resistance (TER). Stable monolayers of ARPE-19 cells grown on transwell plates treated with increasing concentrations of thrombin (in U/mL), which resulted in a time and dose-dependent reduction of TER. Data are plotted as mean±SEM; n=3 independent experiments.

FIGS. 3A-C show thrombin-mediated effects on CTGF and VEGF expression. FIG. 3A show cell lysates that were analyzed for thrombin-mediated CTGF expression. Thrombin induced expression of CTGF that was already maximal at 0.5 U/mL. FIG. 3B show that densitometric analysis demonstrated that thrombin-induced CTGF expression was reduced by dabigatran (10 μM), the protease activated receptor-1 (PAR1) receptor antagonist (SCH79797; 250 μM), and the non-specific protease inhibitor (al-antitrypsin; 1 mg/mL), and was triggered using PAR1-AP (30 μM), a PAR1 agonist. FIG. 3C shows that VEGF secretion (assessed by ELISA) into the apical supernatant was induced by thrombin treatment and reduced by dabigatran and the selective non-peptide PAR1 receptor antagonist SCH79797. VEGF secretion into the apical supernatant was triggered using the PAR1-AP. Data are plotted as mean±SEM; n=3 independent experiments.

FIGS. 4A-B show thrombin-mediated effects on cell-surface activated complement. FIG. 4A shows ARPE-19 cells plated on glass-bottom dishes that were assessed for complement activation based on C3d (green) and MAC (red) deposition on the apical surface. Cell bodies were identified using Hoechst. ARPE cells were treated with serum-free media (control) or with 20 U/mL of thrombin in the presence and absence of dabigatran (10 μM) or TT30 (10 μM). Please note that ARPE-19 cells secrete complement proteins into the supernatant, sufficient to mount a complement attack. FIG. 4B shows C3d and MAC deposits counted by image J were significantly identified in thrombin treated cells and reduced significantly in cells treated with dabigatran or TT30. Data are plotted as mean±SEM; n=3 independent experiments.

FIGS. 5A-C show thrombin-mediated effects on fluid phase-dependent complement activation. FIG. 5A show apical supernatants of treated cells analyzed for thrombin-mediated complement component C3 (left blot) and C5 (right-hand blot) cleavage, using antibodies against C3a and C5a, respectively. FIG. 5B shows reduction of the 120 kDa and generation of an 80 kDa fragment of C3 assessed by densitometric analysis using image J. FIG. 5C shows that the reduction of the 120 kDa band was inhibited by dabigatran (10 μM) and the fluid phase complement inhibitor compstatin (100 μM) (left hand blot) but not the inhibitor of the alternative pathway TT30 (10 μM) that requires membrane binding (right hand blot). Data are plotted as mean±SEM; n=3 independent experiments.

FIGS. 6A-C show that CTGF and VEGF expression can be regulated by complement. FIG. 6A shows that Thrombin-mediated CTGF expression in cells can be inhibited by fluid phase complement inhibition (compstatin). FIG. 6B shows that VEGF secretion into the apical supernatant as measured by ELISA was induced by thrombin, partially reduced by fluid phase or membrane-based complement inhibition (100 μM) and partially induced by C3a receptor activation (C3a, (260 nM). FIG. 6C show that C5a receptor stimulation (C5a, 52 nM) was as potent as thrombin treatment when assessing the amount of VEGF secreted into the apical supernatant. Data are plotted as mean±SEM; n=3 independent experiments.

FIG. 7 shows that dabigatran affects VEGF expression in repair but not angiogenesis phase of CNV. Equal amounts of RPE/choroid extracts (15 μg/lane) were loaded per lane, probed with antibodies as indicated, and band intensities quantified. Arbitrary values were established based on normalization with GAPDH. Age-matched naive animals without CNV and fed normal mouse chow were compared to those in which CNV was and treated with dabigatran during the angiogenesis and repair phase of CNV. During the induction phase and repair phase of CNV, VEGF levels in the RPE/choroid were elevated; however, dabigatran affected VEGF levels in the long-term paradigm.

FIGS. 8A-B show that in the repair phase of CNV, dabigatran reduces CTGF expression. Western blot analysis was performed as described in legend for FIG. 7. FIG. 8A shows that dabigatran reduced CTGF in the wound healing paradigm when compared to the control-treated and naïve animals. FIG. 8B shows that complement activation was increased in RPE/choroid of CNV animals when compared to naïve animals. Surprisingly, complement activation was further increased by treatment when compared to PBS controls. Data are expressed as mean±SEM (n=3-5 independent samples per condition).

FIG. 9 is a summary figure showing that thrombin regulates a dual-signaling mechanism, a) by cleaving C3 and C5, it activates the complement cascade, and b) it modulates down-stream signaling via membrane-bound receptor PAR1. Inhibitors are presented in red and pathways are presented in blue. The study concluded that thrombin induces complement and CTGF, which subsequently activates VEGF secretion in ARPE-19 cells, resulting in disassembly of tight junctions and TER reduction.

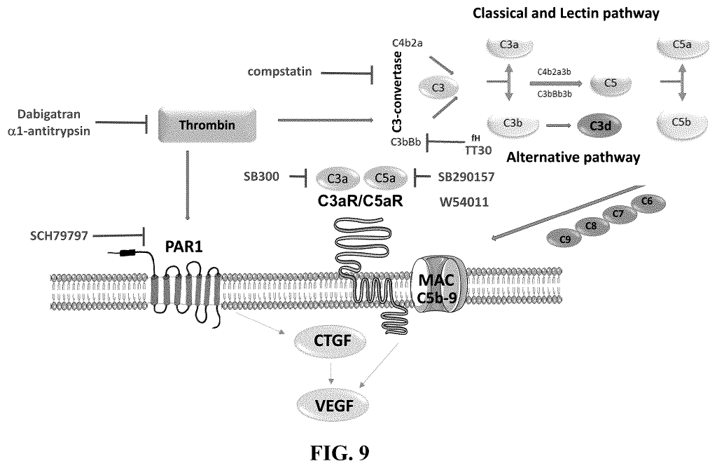

FIGS. 10A-B show that thrombin activity is not affected by complement inhibitors. FIG. shows thrombin activity assessed in a cell-free system. Cleavage of thrombin-specific substrate is proportionate to the concentration of thrombin (10 mU/ml-500 mU/ml) added, and was blocked by the thrombin inhibitor dabigatran. FIG. 10B shows that the alternative pathway inhibitor (TT30) that acts on cell membranes, and the C3-convertase blocker compstatin, which blocks complement both on membranes and in fluid-phase, do not inhibit thrombin activity.

FIG. 11 shows the immunogenicity and molecular weight of the C3a fragment generated by thrombin mediated C3 cleavage. Supernatants of cells treated with thrombin or TT30 were run in parallel with purified mouse C3a (Comptech). The low molecular weight bands produced in the presence of thrombin run at the same molecular weight as purified C3a.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for age-related macular degeneration, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be age-related macular degeneration.

As used herein, the term “inhibit” or “inhibiting” mean decreasing, reducing, or blocking thrombin activity.

Age-related macular degeneration (AMD) is associated with an irreversible destruction of the macula and is the leading cause of visual impairment and irreparable blindness among older population in the Western world (Ferris, F. L., 3rd, et al., Ophthalmology, 2013. 120(4): p. 844-51; Gallego-Pinazo, R., et al., Arch Soc Esp Oftalmol, 2017. 92(2): p. 71-77; and Sun, Z. and Y. Sun, J Biomed Opt, 2019. 24(5): p. 1-9). Around 196 million people worldwide and 11 million in the United States are experiencing either forms of AMD, dry (non-exudative) or wet (exudative). The overall prevalence of the disease is expected to be nearly double by 2050, therefore, imposing a substantial burden on the health care system (Friedman, D. S., et al., Arch Ophthalmol, 2004. 122(4): p. 564-72; and BrightFocus Foundation. Age-Related Macular Degeneration: Facts & Figures. Jan. 5, 2016; Available from: brightfocus.org/macular/article/age-related-macular-facts-figures). The two forms of AMD share characteristics, such as the destruction of the blood-retinal barrier (BRB), development of drusen, activation of immune reposes, and deposition of complement components in the retinal pigment epithelium-bruch's membrane complex (RPE/BrM) (Corbelli, E., et al., Invest Ophthalmol Vis Sci, 2019. 60(5): p. 1394-1402; and Zajac-Pytrus, H. M., et al., Adv Clin Exp Med, 2015. 24(6): p. 1099-104). In the dry form of the disease, these damages can progress to geographic atrophy (GA) that leads to destruction of RPE and choriocapillaris followed by loss of the overlying photoreceptors, whereas in the wet form they ultimately lead to the formation of VEGF-dependent choroidal neovascularization (CNV). In both cases, with the destruction of the BRB, blood-derived factors are likely to get access to the retina and the apical side of the RPE. Thrombin (THR) is likely to get access to those structures upon BRB integrity loss. Therefore, approaches targeting THR are needed.

One of the blood-derived systems that has gained a lot of attention in AMD is the complement system. The complement system is a component of the innate and adaptive immune system. Histological and functional studies have shown the deleterious effects of complement activation and deposition in on RPE health, as well as AMD risk and progression (Morgan, B. P. and C. L. Harris, Nat Rev Drug Discov, 2015. 14(12): p. 857-77; Kumar-Singh, R., Exp Eye Res, 2019. 184: p. 266-277; Rohrer, B., et al., Mol Vis, 2019. 25: p. 79-92; Libby, R. T. and D. B. Gould, Adv Exp Med Biol, 2010. 664: p. 403-9; and Troutbeck, R., et al., Clin Exp Ophthalmol, 2012. 40(1): p. 18-26). Additionally, a genome-wide association study on European-American descent complied significant evidence between factor H polymorphism and increase disease risk of AMD (Hageman, G. S., et al., Proc Natl Acad Sci USA, 2005. 102(20): p. 7227-32). The complement system or cascade is characterized by the generation of a sequential set of proteases that generate biological effector molecules. In short, it is triggered by three distinct pathways: the classical pathway (CP), lectin pathway (LP), and alternative pathway (AP), which contribute to the formation of C3 convertases that cleave the complement component 3 (C3) into C3a and C3b. C3b then participates in the formation of a C5 convertase that cleaves complement component 5 (C5) into C5a and C5b. Finally, C5b initiates the formation of the membrane attack complex (MAC) on membranes resulting in sublytic cell signaling or cell lysis (Sun, Z., and Sun, Y. (2019) J Biomed Opt 24, 1-9). C3a and C5a are anaphylatoxins that participate in different mechanisms, including enhancing vascular permeability and mediating chemotaxis and inflammation (4), by binding to their receptors C3aR, C5aR and C5L2 (BrightFocus Foundation. (Jan. 5, 2016) Age-Related Macular Degeneration: Facts & Figures).

Another blood-derived system is the coagulation system, with its enzyme thrombin (coagulation factor II). Thrombin's main role is to convert soluble fibrinogen into insoluble strands of fibrin as part of the clotting cascade. However, in different models, it is also a known regulator of the destruction of the blood-retinal barrier; it promotes vascular endothelial growth factor (VEGF) secretion, and plays a role in ER-stress induction (Dupuy, E., et al., J Thromb Haemost, 2003. 1(5): p. 1096-102; Machida, T., et al., PLoS One, 2017. 12(5): p. e0177447; and Atanelishvili, I., et al., American journal of respiratory cell and molecular biology, 2014. 50(5): p. 893-902). In C3-deficient mice, for example, thrombin can substitute for the C3-dependent C5 convertase, generating C5a fragments, that are effective as anaphylatoxins (Huber-Lang, M., et al., Nat Med, 2006. 12(6): p. 682-7). Also, thrombin has been shown to uniquely cleave the complement component C5 in vitro, supporting the terminal complement cascade, and resulting in erythrolytic activity (Krisinger, M. J., et al., Blood, 2012. 120(8): p. 1717-25). Proteomics analysis by LC-MS/MS showed that vitreous fluid obtained from AMD patients contains higher amounts of prothrombin compared with healthy controls (Koss, M. J., et al., PLoS One, 2014. 9(5): p. e96895). While no mutations have yet been reported as a risk factor for AMD, two single nucleotide polymorphisms (SNPs), the A-allele of factor V Leiden 1691 or the prothrombin 20210 gene have been found to expose the wet AMD carriers to a higher risk of failing therapeutic effectiveness of the photodynamic therapy with verteporfin (Parmeggiani, F., et al., Recent Pat DNA Gene Seq, 2009. 3(2): p. 114-22).

AMD is a major cause of blindness in the Western world, and one of the reasons is the continued lack of therapeutics other than anti-VEGF medications to block or cure the disease. In addition, a lack of understanding of the pathogenesis of the disease limits novel drug target identification and treatment options offered to the patients. In order to expand the effective treatment options, understanding of the interrelationship or cross-talk between different signaling pathways is of paramount importance. The roles of complement and VEGF in AMD pathogenesis and progression are well studied and accepted as therapeutic targets. Some of the characteristics of AMD, such as, blood-retinal barrier destruction and vascular endothelial growth factor (VEGF) secretion as well as production of anaphylatoxins fit well within the known physiological functions of thrombin. However, little is known about the pathological effects of thrombin in complement activation, VEGF-secretion, and physiological consequences in RPE cells.

Described herein are the multifaceted regulatory roles of thrombin in complement activation and VEGF secretion using an in-vitro model of AMD, RPE cell monolayer. The cross-talk between the complement and the coagulation system was probed with a direct thrombin-inhibitor, dabigatran (New Drug Application: 022512), a membrane targeted alternative pathway of complement inhibitor, CR2-fH/TT30, and a C3 convertase inhibitor that is active in fluid phase, compstatin. Dabigatran (PRADAXA), a direct thrombin-inhibitor, is an anticoagulant used in the treatment and prevention of deep vein thrombosis (DVT) and pulmonary embolism (PE). It achieves its effects by preventing blood clots from forming. It is approved by the FDA for the treatment of deep vein thrombosis and pulmonary embolism in patients who have been treated with a parenteral anticoagulant for five to ten days, and to reduce the risk of recurrent DVT and PE. Disclosed herein is evidence that dabigatran inhibits complement activation on ARPE-19 cell membranes. Also disclosed herein is evidence that dabigatran can inhibit the expression of connective tissue growth factor.

It was also tested whether a correlation exists between dabigatran use and AMD through use of retrospective data from the MarketScan® medical billing record database for Medicare patients. These results were followed up in the mouse model of wet AMD, in which laser photocoagulation of BrM is used to trigger angiogenesis and fibrosis (Rohrer et al., 2009; Parsons et al., 2019); and potential mechanisms of action were further explored in ARPE-19 cell monolayers. Overall, the results described herein demonstrate that subjects who received dabigatran had longer time elapsing than the control population before they had the first recorded diagnosis of AMD. In the mouse model of wet AMD, dabigatran reduced CNV lesion size and accelerated repair processes, a process that seems to involve connective tissue growth factor (CTGF), a known regulator of fibrosis and signaling of growth factors, including VEGF. It was also tested whether thrombin induces complement activation on retinal pigment epithelial cells amplified by the alternative pathway of complement, resulting in VEGF secretion. The results described herein show that therapeutic intervention with the direct thrombin inhibitor-dabigatran would ameliorate disease outcomes in AMD (e.g., wet AMD).

Thrombin is a multifaceted, dynamic enzyme with both coagulant and anticoagulant functions. Thrombin is in the serine protease family. It has three binding domains in which thrombin-inhibition drugs bind. Those proteases have a deep narrow gap as an active binding site that consists of two β-barrel subdomains that make up the surface gap which binds substrate peptides. The surface in the gap seems to have limiting access to molecules by steric hindrance; this binding site consists of three amino acids, Asp-102, His-57 and Ser-195. Thrombin also has two exosites (1 and 2). Thrombin is a little different from other serine proteases as exosite 1 is anion-binding and binds to fibrin and other similar substrates while exosite 2 is a heparin-binding domain. Thrombin functions via proteolytic mechanism as it cleaves the peptide bond between Arg and Ser of the N-terminus of its' target receptor. The cleavage by thrombin thus unmasks the tither ligand of the receptor and can activate downstream signaling.

Direct thrombin inhibitors (DTIs) are a class of anticoagulant drugs that can be used to prevent and treat embolisms and blood clots caused by various diseases; binding directly to thrombin and blocking its interaction with its substrates as well as blocking the cleavage-mediated receptor activation and subsequent cell signaling cascade.

DTIs can inhibit thrombin by two ways; bivalent DTIs block simultaneously the active site and exosite 1 and act as competitive inhibitors of fibrin, while univalent DTIs block the active site, and can therefore both inhibit unbound and fibrin-bound thrombin. In contrast, heparin drugs bind in exosite 2 and form a bridge between thrombin and antithrombin, an anticoagulant substrate formed in the body, and strongly catalyzes its function. Heparin can also form a bridge between thrombin and fibrin which binds to exosite 1 which protects the thrombin from inhibiting function of heparin-antithrombin complex and increases thrombin's affinity to fibrin. DTIs that bind to the anion-binding site have shown to inactivate thrombin without disconnecting thrombin from fibrin, which points to a separate binding site for fibrin. DTIs are not dependent on cofactors like antithrombin to inhibit thrombin so they can both inhibit free/soluble thrombin as well as fibrin bound thrombin unlike heparins. The inhibition is either irreversible or reversible. Reversible inhibition is often linked to a lesser risk of bleeding. Due to this action of DTIs, they can both be used for prophylaxis as well as treatment for embolisms/clots.

Current anticoagulant therapy includes the use of indirect thrombin inhibitors (e.g., heparins, low-molecular-weight-heparins) and vitamin K antagonists such as warfarin. Several caveats exist, however, in the clinical use of these agents including but not limited to narrow therapeutic window, parenteral delivery, and food- and drug-drug interactions. Dabigatran is a synthetic, reversible DTI with high affinity and specificity for its target binding both free and clot-bound thrombin, and offers a favorable pharmacokinetic profile. Large randomized clinical trials have demonstrated that dabigatran provides comparable or superior thromboprophylaxis in multiple thromboembolic disease indications compared to standard of care.

As such, direct thrombin-inhibitors can be categorized as a bivalent inhibitor, an univalent inhibitor or an allosteric inhibitor based on their interaction with thrombin. Examples of bivalent direct thrombin-inhibitors include but are not limited to hirudin, bivalirudin, lepirudin and desirudin. Examples of univalent direct thrombin-inhibitors include but are not limited to argatroban, inogatran, melagatran, ximelagatran and dabigatran. Examples of allosteric direct thrombin-inhibitors include but are not limited to DNA apatmers, benzofuran dimers, benzofuran trimers, polymeric lignins and sulfated β-O4 lignin.

Methods of Treatment

Disclosed herein, are methods of treating age-related macular degeneration in a subject. Disclosed herein, are methods of treating age-related macular degeneration in a subject, the methods comprising: administering to a subject in need thereof a therapeutically effective amount of a direct thrombin-inhibitor. Disclosed herein, are methods of treating age-related macular degeneration in a subject, the methods comprising: (a) identifying a subject in need of treatment; and (b) administering to the subject a therapeutically effective amount of a direct thrombin-inhibitor. In some aspects, the direct thrombin-inhibitor can be dabigatran. In some aspects, the administration of the direct thrombin-inhibitor (e.g., dabigatran) can reduce complement activation, C3d deposits, membrane attack complex of complement deposits or a combination thereof in the subject. In some aspects, the administration of the direct thrombin-inhibitor (e.g., dabigatran) can decrease the expression of connective tissue growth factor in the subject. In some aspects, the methods can comprise administering to a subject in need thereof a therapeutically effective amount of one or more direct thrombin-inhibitors.

Disclosed herein are methods of inhibiting expression of connective tissue growth factor in a subject. Disclosed herein are methods of inhibiting expression of connective tissue growth factor in a subject, the methods comprising: administering to the subject in need thereof a therapeutically effective amount of a direct thrombin-inhibitor. Disclosed herein are methods of inhibiting expression of connective tissue growth factor in a subject, the methods comprising: (a) identifying a subject in need of treatment; and (b) administering to the subject a therapeutically effective amount of a direct thrombin-inhibitor. The methods of inhibiting expression of connective tissue growth factor in a subject can include contacting a cell or tissue or administering to a subject in need thereof, a therapeutically effective amount of a direct thrombin-inhibitor. In some aspects, the direct thrombin-inhibitor can be dabigatran. In some aspects, the complement activation can be decreased. In some aspects, the methods can comprise administering to a subject in need thereof a therapeutically effective amount of one or more direct thrombin-inhibitors.

Disclosed herein are methods of reducing complement activation, C3d deposits and/or membrane attack complex of complement (MAC) deposits in a cell, a tissue, a membrane or an extracellular matrix in a subject. The methods can include contacting a cell or a tissue or administering to a subject in need thereof, a therapeutically effective amount of a direct thrombin-inhibitor. For example, disclosed herein are methods of reducing complement activation, C3d deposits and/or membrane attack complex of complement (MAC) deposits in a cell, a tissue, a membrane or an extracellular matrix, the methods comprising: contacting a cell or a tissue or administering to a subject in need thereof, a therapeutically effective amount of a direct thrombin-inhibitor. In some aspects, the direct thrombin-inhibitor can be dabigatran. In some aspects, the complement activation can be decreased. In some aspects, the methods can comprise administering to a subject in need thereof a therapeutically effective amount of one or more direct thrombin-inhibitors.

Disclosed herein are methods of ameliorating one or more symptoms of age-related macular degeneration in a subject. For example, disclosed herein are methods of ameliorating one or more symptoms of age-related macular degeneration in a subject, the methods comprising: administering to the subject in need thereof a therapeutically effective amount of a direct thrombin-inhibitor. In some aspects, the methods can comprise administering to a subject in need thereof a therapeutically effective amount of one or more direct thrombin-inhibitors.

The methods described herein can comprise administering to a subject in need thereof, a therapeutically effective amount of a direct thrombin-inhibitor. In some aspects, the methods can comprise administering to a subject in need thereof a therapeutically effective amount of one or more direct thrombin-inhibitors. For example, disclosed herein are methods of ameliorating one or more symptoms of age-related macular degeneration in a subject, the methods comprising: (a) identifying a subject in need of treatment; and (b) administering to the subject a therapeutically effective amount of a direct thrombin-inhibitor. In some aspects, the direct thrombin-inhibitor can be dabigatran. In some aspects, the one or more symptoms of age-related macular degeneration can include any of the following: blurry or fuzzy vision, difficulty recognizing familiar faces, straight lines appear wavy, a dark, empty area or blind spot in the center of vision, loss of central vision (important for driving, reading, recognizing faces and performing close-up work), and the presence of drusen (tiny yellow deposits in the retina). In some aspects, the complement activation can be decreased.

In some aspects, the direct thrombin-inhibitor used or administered in the methods disclosed herein can be any direct thrombin-inhibitor. In some aspects, one or more direct thrombin-inhibitors can be administered in the methods disclosed herein. Direct thrombin inhibitors (DTIs) are a class of anticoagulant drugs that can be used to prevent and treat embolisms and blood clots caused by various diseases; binding directly to thrombin and blocking its interaction with its substrates. Direct thrombin-inhibitors can be categorized as a bivalent inhibitor, an univalent inhibitor or an allosteric inhibitor based on their interaction with thrombin. Examples of bivalent direct thrombin-inhibitors include but are not limited to hirudin, bivalirudin, lepirudin and desirudin. Examples of univalent direct thrombin-inhibitors include but are not limited to argatroban, inogatran, melagatran, ximelagatran and dabigatran. Examples of allosteric direct thrombin-inhibitors include but are not limited to DNA apatmers, benzofuran dimers, benzofuran trimers, polymeric lignins and sulfated β-O4 lignin.

Therapeutic administration of any of the direct thrombin-inhibitors encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of age-related macular degeneration.

The direct thrombin-inhibitors described herein can be administered to the subject (e.g., a human patient) in an amount sufficient to delay, reduce, or preferably prevent the onset of clinical disease. Accordingly, in some aspects, the subject is a human subject. In therapeutic applications, compositions are administered to a subject (e.g., a human patient) already with or diagnosed with or at risk for age-related macular degeneration in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition comprising a direct thrombin-inhibitor (e.g., a pharmaceutical composition) can be an amount that achieves a cure, but that outcome is only one among several that can be achieved. As noted, a therapeutically effective amount includes amounts that provide a treatment in which the onset or progression of the age-related macular degeneration is delayed, hindered, or prevented, or the age-related macular degeneration or a symptom of the age-related macular degeneration is ameliorated. One or more of the symptoms can be less severe. Recovery can be accelerated in an individual who has been treated.

In some aspects, the age-related macular degeneration can be a dry form or a wet form. In some aspects, the age-related macular degeneration can be a dry form. In some aspects, the age-related macular degeneration can be a wet form. The dry form of age-related macular degeneration is the most common type of age-related macular degeneration. While the exact cause is unknown, genetic and environmental factors likely play a role. This happens as the light-sensitive cells in the macula slowly break down, generally one eye at a time. The loss of vision in this condition is usually slow and gradual. It is believed that the age-related damage of an important support membrane under the retina contributes to dry age-related macular degeneration. The wet form is less common, and usually leads to more severe vision loss in patients than dry form of age-related macular degeneration. It is the most common cause of severe loss of vision. Wet age-related macular degeneration happens when abnormal blood vessels start to grow beneath the retina. They leak fluid and blood and can create a large blind spot in the center of the visual field.

Disclosed herein, are methods of reducing the risk of a wet AMD in a subject. Disclosed herein, are methods of reducing the risk of a wet AMD diagnosis in a subject. In some aspects, the methods can comprise: administering to the subject in need thereof a therapeutically effective amount of a direct thrombin-inhibitor. Disclosed herein are methods of reducing the risk of a wet AMD diagnosis in a subject, the methods comprising: (a) identifying a subject in need of treatment; and (b) administering to the subject a therapeutically effective amount of a direct thrombin-inhibitor. The methods of reducing the risk of a wet AMD in a subject or reducing the risk of a wet AMD diagnosis can include contacting a cell or tissue or administering to a subject in need thereof, a therapeutically effective amount of a direct thrombin-inhibitor. In some aspects, the direct thrombin-inhibitor can be dabigatran. In some aspects, the methods can comprise administering to a subject in need thereof a therapeutically effective amount of one or more direct thrombin-inhibitors.

Disclosed herein, are methods of treating a subject with age-related macular degeneration. In some aspects, the subject has been diagnosed with age-related macular degeneration prior to the administering step. To diagnose age-related macular degeneration, one or more of the following tests can be carried out: visual acuity test (common eye chart test) that measures vision ability at various distances; pupil dilation; fluorescein angiography (e.g., can be used to detect wet form of age-related macular degeneration); and Amsler grip (e.g., can be used to detect wet form of age-related macular degeneration) which uses a checkerboard like grid to determine if the straight lines in the pattern appear wavy or are missing to the subject. In some aspects, one or more of the following assessments can be carried out alone or in combination with each other and/or the tests provided above: determining the drusen load and size; Choroidal neovascularization (CNV); edema, outer retinal thinning; Bruch's membrane thickening and the like.

In some aspects, the methods of treating a subject with age-related macular degeneration can further comprise reducing or ameliorating one or more symptoms of age-related macular degeneration. In some aspects, the administration of dabigatran can reduce or ameliorate one or more symptoms of age-related macular degeneration. Examples of age-related macular degeneration symptoms can include any of the following: blurry or fuzzy vision, difficulty recognizing familiar faces, straight lines appear wavy, a dark, empty area or blind spot in the center of vision, loss of central vision (important for driving, reading, recognizing faces and performing close-up work), and the presence of drusen (tiny yellow deposits in the retina). In some aspects, the presence of drusen can mean that a subject is at risk for developing a severe type of age-related macular degeneration.

Any of the direct thrombin-inhibitors described herein can be formulated to include a therapeutically effective amount alone or in combination with one or more other therapeutic agents (e.g., an anti-VEGF therapeutic, a complement therapeutics or an oxidant). In some aspects, dabigatran can be contained within a pharmaceutical formulation. In some aspects, the pharmaceutical formulation can be a unit dosage formulation.

The therapeutically effective amount or dosage of any of the direct thrombin-inhibitors (e.g., dabigatran) used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the age-related macular degeneration symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of dabigatran can be in the range of 20 mg to 300 mg/day. In some aspects, the dosage of dabigatran can be 20, 30, 40, 50, 75, 110, 150, or 300 mg total or any amount in between. In some aspects, the dosage of dabigatran can be 20, 30, 40, 50, 60, 75, 80, 100, 110, 120, 150, 160, 220, or 300 mg total or any amount in between. In some aspects, the therapeutically effective dose of dabigatran may be less when combined with one or more of the compounds disclosed herein. In some aspects, dosages of dabigatran can be in the range of 20, 40, 50, 75, 110, or 150 mg twice a day.

In some aspects, dabigatran can be formulated in a self-nanoemusifying drug delivery system (e.g., dabigatran etexilate (DE)-loaded self-nanoemulsifying drug delivery system). In some aspects, dabigatran can be delivered as a nanoparticle ocularly. Examples of formulations of dabigatran and delivery modulates can be found in Chai et al. (Nanomedicine, Vol. 11, No. 14, 2016); Jiang et al. (Int J Ophthalmol. 2018; 11(6): 1038-1044); and Kim and Woo (Pharmaceutics, 2021; 13(1): 108); each of these references are incorporated by reference.

The total effective amount of the direct thrombin-inhibitors (e.g., dabigatran) as disclosed herein can be administered to a subject as a single dose, either as a bolus or by infusion over a relatively short period of time, or can be administered using a fractionated treatment protocol in which multiple doses are administered over a more prolonged period of time.

The direct thrombin-inhibitors described herein can be administered in conjunction with other therapeutic modalities to a subject in need of therapy. The present direct thrombin-inhibitors can be given to prior to, simultaneously with or after treatment with other agents or regimes. For example, dabigatran alone or with any of the therapeutic agents disclosed herein can be administered in conjunction with standard therapies used to treat age-related macular degeneration. In some aspects, any of the direct thrombin-inhibitors described herein can be administered or used together with an anti-VEGF therapeutic, a complement therapeutic, an antioxidant, or a combination thereof. In some aspects, any of the direct thrombin-inhibitors described herein can be co-formulated with an anti-VEGF therapeutic, a complement therapeutic, an antioxidant, or a combination thereof.

Any of the direct thrombin-inhibitors described herein can be administered alone, in combination with another direct thrombin-inhibitor, or as a “combination” with an anti-VEGF therapeutic, a complement therapeutic, an antioxidant, or a combination thereof. It is to be understood that, for example, dabigatran can be provided to the subject in need, either prior to administration of an anti-VEGF therapeutic, a complement therapeutic, an antioxidant or any combination thereof, concomitant with administration of said anti-VEGF therapeutic, a complement therapeutic, an antioxidant or any combination thereof (co-administration) or shortly thereafter.

Pharmaceutical Compositions

Pharmaceutical compositions comprising a direct thrombin-inhibitor can be used in the methods disclosed herein. As disclosed herein, are pharmaceutical compositions comprising a direct thrombin-inhibitor as disclosed herein. As disclosed herein, are pharmaceutical compositions, comprising a direct thrombin-inhibitor as described herein and a pharmaceutical acceptable carrier. In some aspects, the pharmaceutical compositions can include any of the direct thrombin-inhibitors described herein co-formulated with an anti-VEGF therapeutic, a complement therapeutic, an antioxidant, or a combination thereof. In some aspects, the direct thrombin-inhibitor can be dabigatran. In some aspects, the direct thrombin-inhibitor can be formulated for oral or parenteral administration. In some aspects, the parenteral administration can be intravenous, subcutaneous, intramuscular or direct injection. In some aspects, the route of administration can be an intravitreal injection. In some aspects, the route of administration can be an eye drop administration. In some aspects, the route of administration can be carried out via a depot for slow release of the direct thrombin-inhibitor. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions and pharmaceutical compositions disclosed herein can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, depots, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. The pharmaceutical compositions as disclosed herein can be prepared for direct injection, intravitreal injection, eye drop administration, and administration via a depot. Thus, compositions can be prepared for parenteral administration that includes dabigatran or any of the direct thrombin-inhibitors dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, a pharmaceutical composition comprises a direct thrombin-inhibitor and optionally, a pharmaceutical acceptable carrier. Further, the pharmaceutical composition comprises a direct thrombin-inhibitor in a therapeutically effective amount. In some aspects, the direct thrombin-inhibitor can be a bivalent inhibitor, a univalent inhibitor or an allosteric inhibitor. In some aspects, the bivalent inhibitor bivalent direct thrombin-inhibitors can be hirudin, bivalirudin, lepirudin or desirudin. In some aspects, the univalent inhibitor bivalent direct thrombin-inhibitors can be argatroban, inogatran, melagatran, ximelagatran or dabigatran. In some aspects, the allosteric inhibitor can be a DNA apatmer, a benzofuran dimer, a benzofuran trimer, a polymeric lignin, or sulfated β-O4 lignin.

Articles of Manufacture

The compositions and pharmaceutical compositions disclosed herein can be packaged in a suitable container labeled, for example, for use as a therapy to treat age-related macular degeneration or any of the methods disclosed herein. Accordingly, packaged products (e.g., sterile containers containing the composition described herein and packaged for storage, shipment, or sale at concentrated or ready-to-use concentrations) and kits, including at least a direct thrombin-inhibitor as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: Cross-Talk Between the Complement and Coagulation System in Retinal Pigmented Epithelial (RPE) Cell Monolayers

Age-related macular degeneration (AMD), the leading cause of irreversible blindness in elderly Caucasian populations, includes destruction of the blood-retina barrier (BRB) generated by the retinal pigment epithelium-Bruch's membrane complex (RPE/BrM), and complement activation. Thrombin is likely to get access to those structures upon BRB integrity loss. Described herein is the role of thrombin in AMD by analyzing effects of the thrombin inhibitor dabigatran.

MarketScan data for patients aged ≥65 years on Medicare was used to identify association between AMD and dabigatran use. ARPE-19 cells grown as mature monolayers were analyzed for thrombin effects on barrier function (transepithelial resistance; TER) and downstream signaling (complement activation, expression of connective tissue growth factor (CTGF), and secretion of vascular endothelial growth factor (VEGF)). Laser-induced choroidal neovascularization (CNV) in mouse is used to test the identified downstream signaling.

Risk of new wet AMD diagnosis was reduced in dabigatran users. In RPE monolayers, thrombin reduced TER, generated unique complement C3 and C5 cleavage products, led to C3d/MAC deposition on cell surfaces, and increased CTGF expression via PAR1-receptor activation and VEGF secretion. CNV lesion repair was accelerated by dabigatran, and molecular readouts suggest that downstream effects of thrombin include CTGF and VEGF, but not the complement system.

The results disclosed herein provide evidence of an association between dabigatran use and reduced exudative AMD diagnosis. Based on the cell- and animal-based studies, thrombin modulates wound healing and CTGF and VEGF expression, making dabigatran a treatment option in AMD.

Material and methods. MarketScan analysis: Population. A total of 41,860 dabigatran-exposed and 41,860 non-exposed controls, for a total of 83,720 Medicare patients were extracted from MarketScan® data from 2010-2015 and used in the analysis. Patients with an inpatient or outpatient record with a diagnosis of AMD (wet or dry) for the baseline period were excluded from inclusion in the data set. Patients who filled at least one prescription of dabigatran during the 2011-2012 exposure period were included in the dabigatran group. The date of the first filled prescription was designated as the Index Date. A control group of 1,000,000 patients who had no records of dabigatran prescriptions during the baseline or exposure period were randomly selected from outpatient utilization records. The first patient record date in randomly selected records from 2011 or 2012 was chosen as the Index Date for control group patients. Patients with <90 days of insurance coverage in the baseline period or <120 days of coverage in the study (exposure) period were excluded from the final cohort. The exposed and control patients were matched by age, sex, state of residence, Index Date, days in baseline period, and days in study period using 1:1 propensity score mating with a greedy algorithm and a caliper distance of 0.2. A total of 41,860 exposed patients and similar group of controls selected from a pool of 122,636 patients were matched within the matching specification.

Survival and Statistical Analysis. Survival analysis was used (Schnabolk et al., 2019) to show differences in time until AMD diagnosis among dabigatran users and control subjects using the LIFETEST procedure. AMD diagnoses were identified using ICD-9 codes 362.5 (unspecified macular degeneration), 362.51 (dry AMD), and 362.52 (wet AMD). A period of ˜5 years (from Jul. 1, 2010, to Dec. 31, 2015) was used for this analysis, and preexisting AMD diagnosis was ruled out by requiring that the subjects be AMD free over the first ˜600 days (designated as baseline days or Base Days). Data on the matched control and dabigatran population were analyzed using multivariable logistic and Cox regression. Specifically, the odds of being diagnosed with AMD during the follow-up time, controlling for days of dabigatran exposure, and the proportional hazard rates of obtaining an AMD diagnosis in the dabigatran or control population as well as any baseline differences in demographics or comorbidities was compared (Charlson or Elixhouser diagnoses).

Mouse model of choroidal neovascularization (CNV): Choroidal neovascularization. C57BL/6J mice (males and females) from the same colony, to guarantee identical microbiome (Andriessen et al., 2016), were recruited to the study at 3 months of age. Argon laser photocoagulation (532 nm, 100 μm spot size, 0.1 s duration, 100 mW) was used to generate 4 laser spots around the optic nerve of each eye (Rohrer et al., 2009), utilizing bubble formation at the site of the laser burn as the inclusion criteria for successful Bruch's membrane rupture (Nozaki et al., 2006).

Dabigatran exposure. Mice were fed standard chow fortified with dabigatran etexilate (10 μgig of chow; Boehringer Ingelheim). Mice were either fed dabigatran chow during the angiogenesis period of CNV (starting 2 days prior to CNV induction through day 6 when the tissues were collected), or during the wound healing period (starting after the OCT analysis to ensure CNV growth on day 5 through day 23).

Optical Coherence Tomography (OCT) analysis. OCT was used to quantify CNV lesion size on day 5 post laser treatment (Schnabolk, G., et al. (2014). Exp Eye Res 129, 18-23; Schnabolk, G., et al. (2015). Invest Ophthalmol Vis Sci; and Coughlin, B., et al. (2016). Sci Rep 6, 23794), or in weekly intervals to document recovery from fibrosis (Parsons, N., et al. (2019). Mol Immunol 108, 8-12) using an SD-OCT Bioptigen® Spectral Domain Ophthalmic Imaging System (Bioptigen Inc., Durham NC). Mice were anesthetized prior to imaging, eyes kept hydrated with normal saline, and body temperature was carefully monitored to prevent anesthesia-induced cataracts. The methods for imaging and analysis have been described (Schnabolk, G., et al. (2014). Exp Eye Res 129, 18-23; Schnabolk, G., et al. (2015). Invest Ophthalmol Vis Sci; Coughlin, B., et al. (2016). Sci Rep 6, 23794; and Parsons, N., et al. (2019). Mol Immunol 108, 8-). Based on the size of the individual pixels (1.6×1.6 μm), the lesion sizes were calculated.

Cell culture experiments: Cells. Human ARPE-19 (ATCC® CRL-2302™; American Type Culture Collection, Manassas VA) cells were cultured in DMEM cell culture media (Gibco/ThermoFisher Scientific) containing high glucose DMEM with D-glucose (4.5 g/L), L-glutamine, sodium pyruvate (110 mg/L), Penicillin and Streptomycin (1×) and 10% of FBS. Cells (<passage 10 after purchase) were expanded in T75 cell culture flasks at 37° C., in the presence of 5% CO₂. Confluent cells were trypsinized with 0.05% trypsin (Gibco), and equal numbers of cells were seeded on 6-well transwell filters/plate (Costar). After confluent monolayers of cells were developed, the percentage of FBS was gradually decreased from 10%, 2%, to 1% to promote cell differentiation and tight junction formation. Integrity of the cell monolayer was assessed by Transepithelial resistance (TER) measurements using an EVOM volt-ohmmeter (World Precision Instruments) four weeks after plating. RPE monolayers with a stable TER repeatedly measured as ˜40-45 S2 cm 2 were used for these experiments. In addition to TER, the integrity of the monolayers was confirmed by demonstrating the presence of β-actin filament distribution in the form of circumferential bundles, the presence of two cell-junction markers at the cell-borders, ZO-1 and occludin, and co-labeling of ZO-1 and phalloidin (Obert, E., et al. (2017). J Mol Med (Berl))), and the presence of RPE65 in four-week-old ARPE-19 cell monolayers on transwell plates has been reported (Balmer, D., et al. (2017). Frontiers in Aging Neuroscience 9).

Treatments. Prior to each experiment, the cell monolayer was washed with serum-free medium (SFM) and maintained in SFM for 24 hours. During that time, RPE cells secrete proteins, including complement components into the supernatant (Kunchithapautham, K., et al., J Biol Chem, 2014. 289(21): p. 14534-46; Kunchithapautham, K., et al., Adv Exp Med Biol, 2011. 723: p. 23-30; and Thurman, J. M., et al., J Biol Chem, 2009. 284(25): p. 16939-47). Treatments were performed on the apical side of the transwell membrane, which represents the retinal side of the RPE in vivo. Some of the cells were treated with thrombin (EMD Millipore Corp), complement components C3a, C5a (Complement Technology), thrombin agonist PAR1-AP (Sigma-Aldrich; amino acid sequence SFLLRN (SEQ ID NO: 1)), thrombin inhibitor dabigatran etexilate (Sigma-Aldrich), C3 inhibitor compstatin (R&D Systems), AP inhibitor TT30 (Alexion Therapeutics), a protease inhibitor alpha1-antitrypsin (Sigma-Aldrich), and the thrombin receptor proteinase activated receptor 1 inhibitor SCH 79797 (Sigma-Aldrich). Treatments were performed 60 min prior to the addition of thrombin. Cell culture supernatant and lysate were collected after specific time points or after 24 hrs of treatment and stored at −20° C. for later use.

Western blotting. Mouse RPE/choroid/sclera (from herein referred to as RPE/choroid fraction) preparations were extracted (Annamalai, B., et al. (2021). Invest Ophthal-mol Vis Sci 62, 11). Apical supernatants from ARPE-19 cells were concentrated using Amicon Ultra-4 centrifugal filters (EMD Millipore) at 4° C. Cell lysates were collected in RIPA cell lysis buffer containing 150 mM NaCl, 1.0% IGEPAL® CA-630, 0.5% sodium deoxycholate, 0.1% SDS, 50 mM Tris (pH 8.0) (Sigma-Aldrich) in presence of 1×protease inhibitor (Sigma-Aldrich). Equal amount of each samples were loaded on 4-20% Criterion™ TGX™ precast gels (Bio-Rad Laboratories, Inc.) (Annamalai, B., et al. (2020). Invest Ophthalmol Vis Sci 61, 45). Proteins were transferred to a nitrocellulose membrane and incubated in primary antibody followed by appropriate secondary antibodies coupled to peroxidase, followed by band development and detection using Clarity™ Western ECL blotting substrate (Bio-Rad Laboratories, Inc.) and chemiluminescent detection. Protein bands were scanned and densities quantified using ImageJ software. The following antibodies were used: C3a (1:1000; Complement Technology), C5a (1:1000; Abcam), connective tissue growth factor/CTGF (1:1000; Abcam), C3d anti-C3d (clone 11, 1:1000; generation of the antibody is described in (Thurman, J. M., et al. (2013). J Clin Invest 123, 2218-2230)), VEGF (1:1000, Santa Cruz Biotechnology, Inc.), β-actin (1:2000; Cell Signaling Technology) and concentrations selected based on the data sheet recommendations.

Thrombin activity assay. Thrombin activity was measured according to a methodology described by Ludwicka with modifications (Ohba, T., et al. (1994). Am J Respir Cell Mol Biol 10, 405-412). Thrombin specific peptide Boc-Val-Pro-Arg-7-amido-4-methylcoumarin hydrochloride (Sigma-Aldrich) was reconstituted in molecular grade ice-cold H₂O at a concentration of 0.5 mg/ml and used as substrate. To measure the level of active thrombin in serum or tissue extracts, an aliquot (100 μl) was mixed with 50 μl of assay buffer (50 mM Tris, 100 mM NaCl, and 0.01% BSA; pH 7.5) and 50 μl of thrombin substrate at 37° C. Absorbance was read on a spectrophotometer at 405 nm using a Biotek Synergy HT Microplate Reader and thrombin activity was determined by extrapolation from a thrombin standard curve.

Immunofluorescence staining. ARPE-19 cells were grown on transwell membranes in 6 well plates (Thurman, J. M., et al. (2009). J Biol Chem 284, 16939-16947), treated with thrombin and inhibitors, and fixed in 4% paraformaldehyde for 15 min at room temperature. Primary antibodies against C5b-9 (1:200, Abcam) and C3d (1:100) (Thurman, J. M., et al. (2013). J Clin Invest 123, 2218-2230) were diluted in blocking buffer (10% normal goat serum, 3% BSA, 0.4% Triton X-100 in PBS) and cells were incubated for overnight at 4° C., followed by washes and incubation with goat anti-rabbit Alexa Fluor® 568—conjugated IgG or donkey anti-goat Alexa Fluor® 488-conjugated secondary antibodies (1:500; Thermo Fisher Scientific), respectively. Nuclei were identified with DAPI (Sigma-Aldrich). Transwell membranes were covered with aqua-mount mounting media (Thermo Scientific), cover slipped and photographed (Olympus 1X73 Research Inverted Microscope equipped with cellSens imaging software).

VEGF ELISA. VEGF in the supernatant was measured using the Quantikine® Human VEGF Immunoassay (R & D System) according to the manufacturer's protocol. In brief, 200 μl of standard/sample was mixed with 50 μl of assay diluent and added to the plate precoated with the capture antibody. After washing, 200 μl of the VEGF detection antibody (polyclonal antibody specific for human VEGF conjugated to horseradish peroxidase) was added, followed by freshly prepared substrate reagents (stabilized hydrogen peroxide and tetramethylbenzidine). The reaction was terminated using 50 μl of stop solution and absorbance was measured at λ1 450 nm with wavelength correction λ2 at 540 nm. VEGF concentration of samples were calculated comparing the absorbance with the standards and converted into fold change considering the serum-free condition as 1 in the scale.

Thrombin activity in cell-free system. To measure the level of active thrombin in cell-free system, known concentrations of thrombin were prepared in 100 μl of assay buffer (50 mM Tris, 100 mM NaCl, and 0.01% BSA; pH 7.5), subsequently 50 μl of thrombin substrate was added and incubated at 37° C. Fluorescence was measured at 405 nm using the Biotek Synergy HT Microplate Reader and thrombin activity was determined by extrapolation from a thrombin standard curve. Effects of inhibitors were tested by pre-incubating the inhibitors with thrombin, before substrate incorporation.

Statistics. For data consisting of multiple groups, one-way ANOVA followed by Fisher's post hoc test (P<0.05) was used; single comparisons were analyzed by t test analysis (P<0.05).

Results. Association between Dabigatran Use and AMD Risk. The patient population consisted of 83,720 Medicare patients extracted from MarketScan® data from 2010-2015 as described herein, with 41,860 dabigatran-exposed and 41,860 non-exposed controls. The controls were matched on 33 baseline variables, including demographics and clinical characteristics (Schnabolk, G., et al. (2019). Invest Ophthalmol Vis Sci 60, 3520-3526) (Table 1). Patients who received dabigatran had a longer time elapsing than the control population (P<0.0001) before they had the first recorded diagnosis of unspecified AMD (hazard ratio 0.63). Overall, the percent subjects with an AMD diagnosis was 13.95% in the control group, and reduced to 9.1% in the dabigatran group, with the percent AMD per study year being reduced by ˜35%. Specifically, the percent patients with a wet AMD diagnosis was reduced by ˜60% (control 2.29; dabigatran 0.91) which is reflected in the percent wet AMD per study year (control 0.68; dabigatran 0.28) (Table 2). The percent wet AMD per study year was not affected by the number of years of dabigatran exposure (<1 year 0.28; 1-2 years 0.26; >2 years 0.29). Survival analysis of days to first AMD event show similar findings. Multivariable logistic regression models controlling for age, sex and days in the study showed an odds ratio (OR) of 0.58 (confidence interval CI 0.55-0.61) of AMD for the dabigatran group compared to controls, with risk of AMD by exposure strata of 0.52 for <1 year, 0.64 for 1-2 years and 0.64 for 2+ years of dabigatran compared to controls.

TABLE 1
Characterization of control and dabigatran-exposed subjects. Subjects (41860)
were identified per group and characterized for age, baseline days (days that subjects had to be
free of AMD diagnosis), study days (days after completion of baseline, during which subjects
were interrogated for occurrence of AMD) and days on dabigatran.

| Dabi | N obs | Variable | n | Mean | std dev |
|---|---|---|---|---|---|
| No | 41860 | AGE | 41860 | 75.3 | 7.5 |
| | | Wet Days | 36865 | 1041 | 624.7 |
| | | Dry Days | 41015 | 982 | 629.8 |
| | | Days to AMD | 5840 | 387 | 380.7 |
| | | Dabi Days | 41860 | 0 | 0 |
| | | Base Days | 41860 | 606 | 300.2 |
| | | Study Days | 41860 | 1089 | 616.1 |
| | | AMD Days | 41860 | 967 | 634.3 |
| Yes | 48160 | AGE | 41860 | 76.2 | 6.9 |
| | | Wet Days | 38399 | 1047 | 619.5 |
| | | Dry Days | 41511 | 1011 | 619.6 |
| | | Days to AMD | 3810 | 562 | 417.3 |
| | | Dabi Days | 41860 | 398 | 327.0 |
| | | Base Days | 41860 | 612 | 270.7 |
| | | Study Days | 41860 | 1080 | 620.4 |
| | | AMD Days | 41860 | 1007 | 619.7 |

TABLE 2
Statistical analysis of AMD and dabigatran use. Subjects were subdivided
into dabigatran use or none, as well as stratified based on years of exposure
to the drug during the study period. Patients who received dabigatran had
a lower incidence of AMD, and developed less nAMD per study year than control
subjects, irrespective of the number of years on the drug.

| Years of | | % patients | | % patients | | |
|---|---|---|---|---|---|---|
| Exposure | Mean (SD) | with an | % AMD | with a wet | % wet | Mean |
| to | days of | AMD | per study | AMD | AMD per | (SD) days |
| dabigatran | dabigatran | diagnosis | year | diagnosis | study year | in study |
| None | 0 | 13.95* | 4.67* | 2.29* | 0.68* | 1090 (616) |
| Dabigatran | 398 (327) | 9.10 | 3.07 | 0.91 | 0.28 | 1080 (620) |
| Dabigatran: | | | | | | |
| <1 year | 153 (105) | 7.51 | 3.04 | 0.75 | 0.28 | 900 (610) |
| 1-2 years | 532 (94) | 10.11 | 3.25 | 0.90 | 0.26 | 1135 (549) |
| 2+ years | 903 (132) | 12.26 | 3.00 | 1.35 | 0.29 | 1492 (491) |

Dabigatran reduces CNV lesion size and accelerates repair. The mouse CNV model is an accepted model for wet AMD, and is characterized by two phases, an injury and angiogenesis phase followed by slow repair. It has been shown that in OCT images, maximum CNV size is observed 5 days after the laser burn, followed by a slow repair (Giani, A., et al. (2011). Invest Ophthalmol Vis Sci 52, 3880-3887). It has also been demonstrated that inhibiting the alternative pathway of complement accelerated fibrotic scar resolution, but that repair required homeostatic levels of the anaphylatoxins C3a and C5a (Parsons, N., et al. (2019). Mol Immunol 108, 8-12).

It was test whether dabigatran would alter the course of angiogenesis or repair, by feeding animals with dabigatran chow during the two different phases. Using chow provided by Boehringer Ingelheim, animals were dosed with dabigatran at ˜10 mg/kg bodyweight per day, resulting in a blood plasma concentration of dabigatran (0.372±0.03 μg/ml) and elongating the coagulation times by 3-fold (Pingel, S., et al. (2014). Arch Med Sci 10, 154-160). This level of dabigatran resulted in thrombin activity based on fluorogenic thrombin specific peptide cleavage (Ohba, T., et al. (1994). Am J Respir Cell Mol Biol 10, 405-412) that was reduced by ˜35% in serum (control: 100±7.6; dabigatran: 65.4±5.1; P=0.003), and ˜10% in retina/RPE/choroid tissues (control: 100±3.1; dabigatran: 91.1±2.6; P=0.04) when compared to control mice.

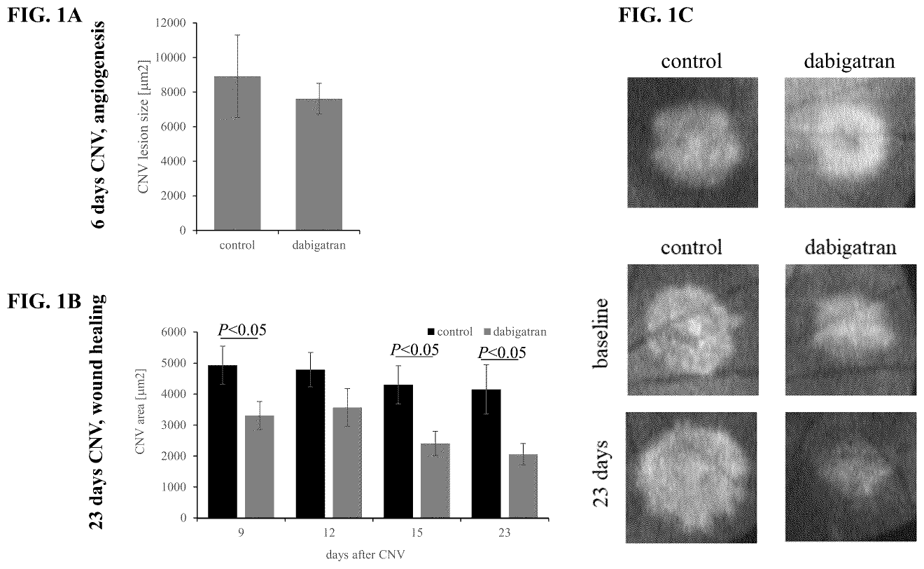

Dabigatran, when provided from 2 days prior to CNV induction through the angiogenesis phase, resulted in a small and insignificant reduction of CNV lesion sizes by ˜15% (P=0.2) (FIG. 1A). In contrast, when dabigatran was provided from day 5 after CNV induction through the repair phase, when analyzing lesion sizes at the final time point on day 23 using post-hoc testing (Fisher's PLSD; StatView, SAS Institute), dabigatran-treated animals had significantly smaller lesions (P<0.02). Since the same animals were followed over a 23-day time course, the treatment responses over time were evaluated using a repeated measure ANOVA. In the analysis of response over time, there was a significant effect of treatment (P<0.0001), which was confirmed by Bonferroni (P<0.0001) (FIG. 1B).

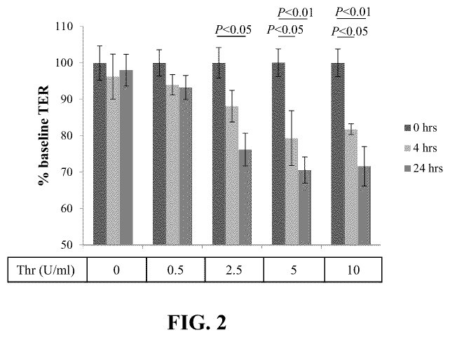

Thrombin reduces barrier function in RPE cells and induces CTGF expression and VEGF secretion. ARPE-19 cells are a good model to test for pathways involved in establishment and loss of barrier function (Ablonczy, Z., and Crosson, C. E. (2007). Exp Eye Res 85, 762-771), and transepithelial resistance (TER) has been found to correlate with VEGF secretion (Kunchithapautham, K., et al. (2011). Adv Exp Med Biol 723, 23-30). Thrombin is a serine-protease and is a known contributor to the damage of the blood-brain barrier (Machida, T., et al. (2017). PLoS One 12, e0177447). To examine the effects of thrombin on the blood-retina barrier (i.e., TER), dose- (0.5-10.0 U/ml) and time- (4 and 24 hrs) dependent treatments were performed on stable ARPE-19 cell monolayers, and treatment effects on TER levels to TER at baseline (serum free media/SFM before treatment, 0 hrs) (FIG. 2) were compared. TER was stable over the 24 hr time course in untreated cells (0 U/mL), and was barely affected by the lowest dose tested (0.5 U/mL). Thrombin at a low concentration (2.5 U/ml) required a longer period of time (24 hrs) to cause significant damage to the epithelial barrier integrity. In contrast, higher concentrations of thrombin (5 U/ml and 10 U/ml) caused significant barrier function loss at the earlier time point (4 hrs).

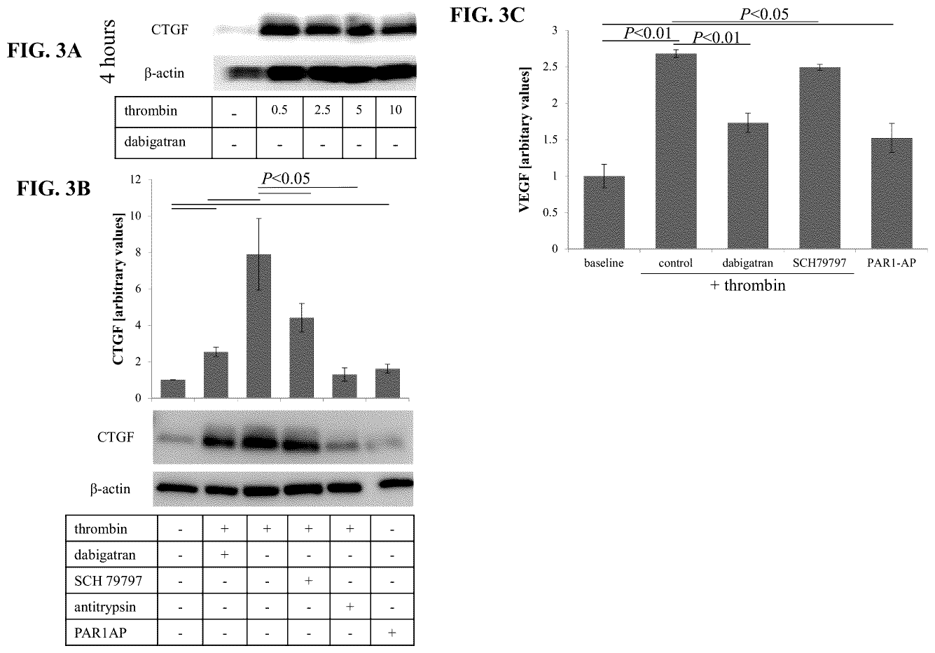

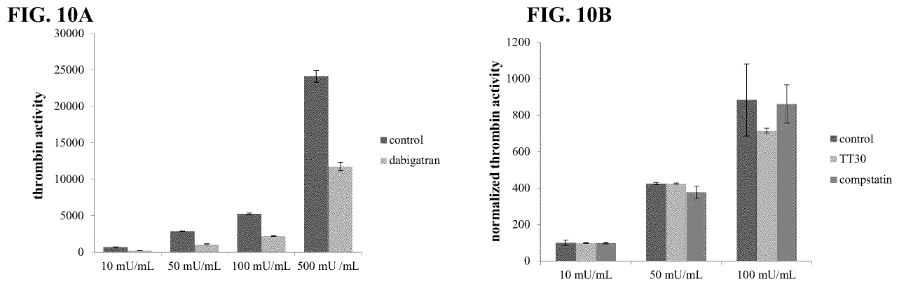

Oxidative stress has been shown to lead to VEGF secretion in ARPE-19 cells (Kunchithapautham, K., et al. (2011). Adv Exp Med Biol 723, 23-30), which in wet AMD is associated with abnormal blood vessel formation (Campa, C., and Harding, S. P. (2011). Curr Drug Targets 12, 173-181). As thrombin has been shown to induce CTGF expression (Ko, W. C., et al. (2012). Acta Pharmacol Sin 33, 49-56) and CTGF is important in the production of VEGF (He, S., et al. (2003). Arch Ophthalmol 121, 1283-1288), it was investigated whether thrombin has similar effects in RPE cells. ARPE-19 cell monolayers were treated with thrombin (0.5-10 U/ml) and cell lysates were collected and analyzed for CTGF levels at 4 hrs post treatment by western blotting. Thrombin was found to increase CTGF expression that, however, did not appear dose-dependent (FIG. 3A). As lower concentrations optimally activated CTGF expression (0.5-2.5 U/mL) and dabigatran (10 μM) is less effective at reducing thrombin activity at higher concentrations (see FIG. 10A), the remaining experiments were performed at 1 U/mL. Thrombin can exert an effect via the thrombin receptor, Protease Activated Receptor-1 (PAR1). This direct thrombin pathway was investigated in cells treated with thrombin in the presence of dabigatran (10 μM), the PAR1 antagonist (SCH79797, 250 μM), the general protease inhibitor alpha1-antitrypsin (1 mg/mL) and the PAR1 activating peptide PAR1-AP (30 μM) (FIG. 3B). Densitometric analyses of the blots from three independent experiments showed that thrombin significantly increased CTGF expression (P<0.05), an effect that was blunted by dabigatran (3.2 fold) and alpha1-antitrypsin (5.3 fold). Specificity for PAR1 was provided by utilizing the PAR1 antagonist SCH79797 (1.7 fold), which partially reduced the expression of CTGF and the PAR1 agonist PAR1-AP which marginally increased the expression (1.5 fold). To investigate whether the levels of CTGF are correlated with VEGF secretion in thrombin-treated ARPE-19 cell monolayers, the apical supernatants from treated monolayers were used for ELISA assays. Thrombin alone resulted in a 2.5-fold increase of VEGF in APRE-19 cell apical supernatants, an effect that was partially blocked by dabigatran, reducing the VEGF content by ˜35% (FIG. 3C). Treatment with the PAR1 agonist increased the secretion of VEGF. Overall, the results suggest that thrombin reduces barrier function in RPE cells in part by stimulating VEGF secretion, an effect that might be mediated in part by PAR1-mediated CTGF expression.

Cross-talk between the complement and coagulation system in ARPE-19 cell monolayers. A second mechanism was investigated. It was previously shown that complement activation on RPE cells impairs barrier function requiring sublytic membrane attack complex (MAC) activation and VEGF secretion (Thurman, J. M., et al. (2009). J Biol Chem 284, 16939-16947), and thrombin has been reported to be able to cleave complement components (Huber-Lang, M., et al. (2006). Nat Med 12, 682-687; and Krisinger, M. J., et al. (2012). Blood 120, 1717-1725). As a prerequisite for the experiments described herein, it was confirmed that dabigatran inhibits thrombin activity in a cell-free system, whereas the two complement inhibitors used here, compstatin and TT30, did not (FIG. 10B).

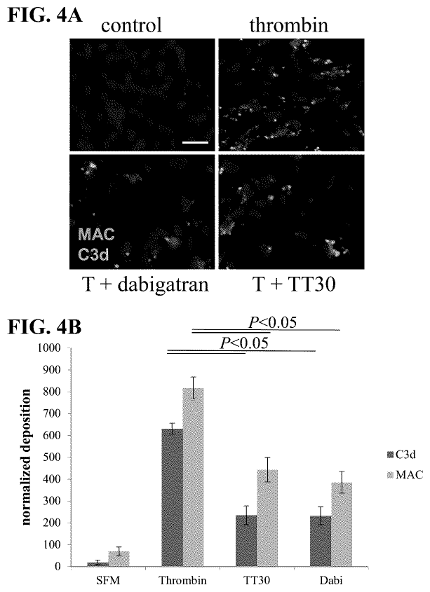

First, it was tested whether thrombin results in deposition of C3d and the assembly of MAC on ARPE-19 cell surfaces. ARPE-19 cell monolayers were treated apically with thrombin and deposition of MAC (C5b-9; red) and C3d (green) were examined by immunofluorescence staining (FIG. 4A). Thrombin treatment induced both C5b-9 and C3d accumulation compared to control (serum free medium; SFM) (FIG. 4A). Both C5b-9 and C3d deposition were reduced with pretreatment of dabigatran (10 μM) (FIG. 4B), or with the alternative pathway of complement inhibitor TT30 (10 μM) (Thurman, J. M., et al. (2009). J Biol Chem 284, 16939-16947) (FIG. 4B). Image analysis showed that dabigatran and TT30 both reduced C3d deposition by ˜60% when compared with thrombin-treated cells. Similarly, C5b-9 deposition was reduced by ˜50% by either of the two inhibitors when compared with thrombin-treated cells. As TT30 inhibits the AP pathway on membranes, this suggests that thrombin activation results in C3b deposition on cell membranes that is amplified by the AP.

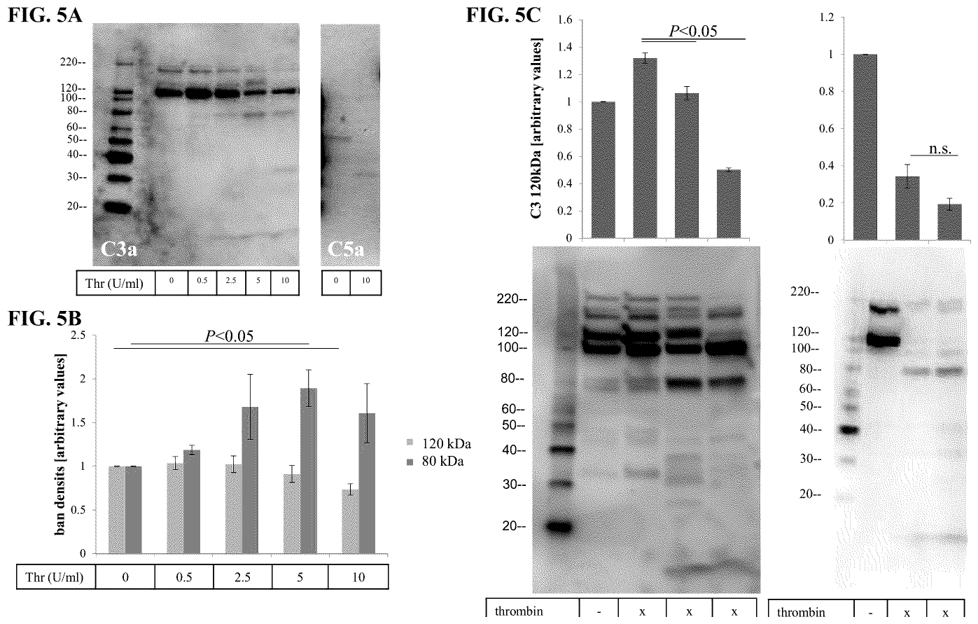

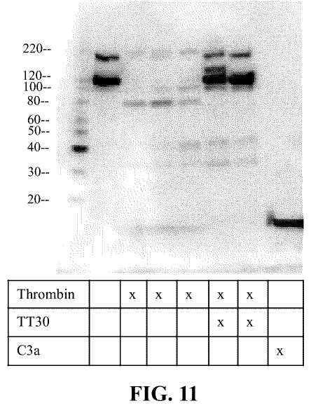

Second, it was tested whether thrombin can cleave complement components (Huber-Lang, M., et al. (2006). Nat Med 12, 682-687; and Krisinger, M. J., et al. (2012). Blood 120, 1717-1725), specifically C3 and C5, producing cleavage products potentially different from those produced by classical convertases. To test the effects of thrombin on complement cleavage, the apical supernatants of treated ARPE-19 cell monolayers were analyzed by western blotting. Cleavage of C3α was evaluated using an antibody specific for C3a (FIG. 5A, left-hand blot). The C3a antibody detected C3, the C3α band as well as other fragments containing C3a. Thrombin caused a concentration dependent cleavage of complement component C3 (˜190 kDa), caused reduction of the 120 kDa C3a band and generation of a new 80 kDa fragment. Fragments at 32 kDa and 9-10 kDa with higher doses of thrombin treatment (FIG. 5A, 10 U/mL) were also observed. The 9-10 kDa band ran at the same molecular weight as purified C3a (FIG. 11). The reduction of the 120 kDa fragment was correlated with the generation of the 80 kDa fragment (FIG. 5B). The loss of the 120 kDa fragment was prevented by either dabigatran (10 μM) or the C3 inhibitor compstatin (100 μM), which inhibits complement both on cell membranes as well as in fluid phase, but not by TT30 (10 μM), which is an inhibitor that prevents membrane-bound AP activation (FIG. 5C). In addition, thrombin on cleavage of complement component C5α could be demonstrated using an antibody against C5a (FIG. 5A, right-hand blot). A unique 35 kDa, C5a-like fragment as previously shown by Krisinger (Krisinger, M. J., et al. (2012). Blood 120, 1717-1725) was also observed.

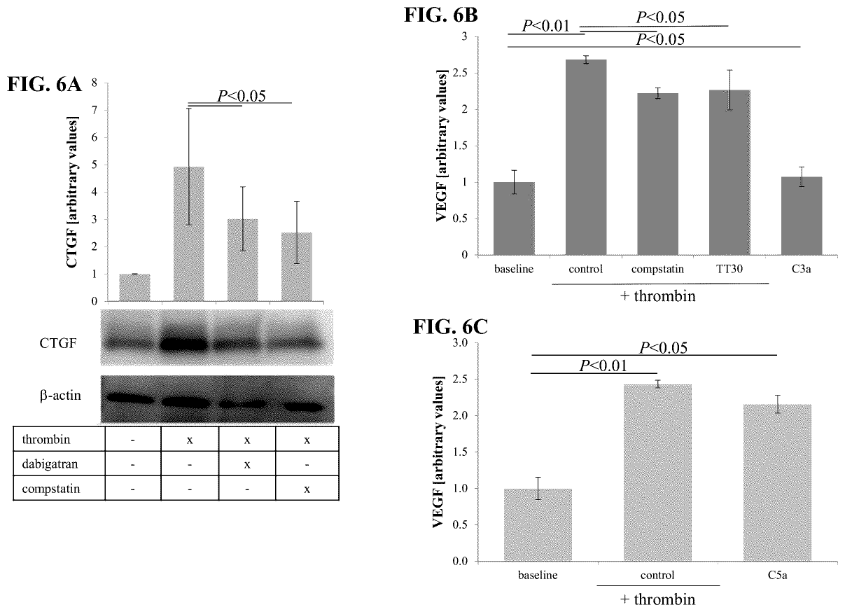

Finally, to tie in CTGF and VEGF, CTGF expression and VEGF secretion were analyzed in response to thrombin and compstatin. Inhibition of complement by compstatin was found to partially reduce thrombin-induced CTGF expression (FIG. 6A) and VEGF secretion (FIG. 6B). Interestingly, a significant driver of VEGF secretion was found to be C5a, increasing VEGF levels to that identified by thrombin stimulation (FIG. 6C), whereas C3a was a weak stimulator. Overall, the data show that thrombin-mediated CTGF expression is partially regulated by complement activation; and VEGF secretion appears to be triggered by thrombin in a combined fashion, involving PAR1 activation, presumably sublytic MAC activation based on the observed MAC deposition and previous data (Thurman, J. M., et al. (2009). J Biol Chem 284, 16939-16947), and C5a receptor activation.

Pathway analysis in CNV. Based on the results in cells, an analysis in animals followed, focusing on VEGF levels in the RPE/choroid, complemented by CTGF analysis and complement activation (C3d production and binding). RPE/choroid samples were collected at the end of the angiogenesis experiment (day 6) and after the final OCT analysis in the repair experiment (day 23) and compared to naive controls (no CNV and no dabigatran treatment).

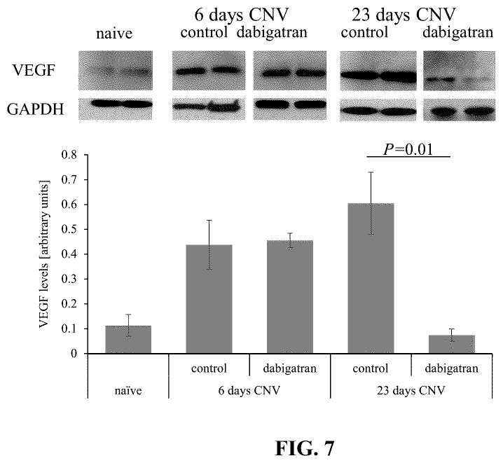

The results shows that VEGF levels were elevated in response to CNV in PBS treated animals 6 days after CNV induction (P=0.03), and remained elevated at the 23-day time point (P=0.01) (FIG. 7). While dabigatran had no effect on VEGF levels at the 6-day time point (P=0.9), dabigatran significantly reduced VEGF levels at the 23-day time point (P=0.001). Dabigatran's effect on VEGF levels were correlated with its effect, or lack thereof, on the size of the CNV lesions at 23 and 6 days, respectively.

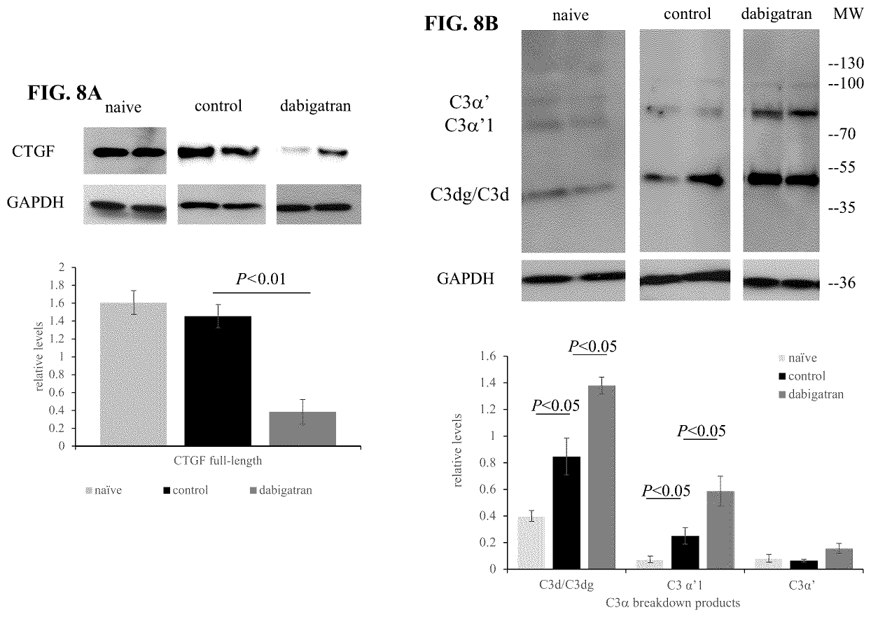

Based on the observed effects of dabigatran on the wound healing component of CNV, these samples were further studies. In the three groups the full-length 38 kDa band of CTGF was present (FIG. 8A). At the end of the repair period (23 days), levels were indistinguishable between naïve and control food CNV animals, whereas dabigatran significantly reduced the levels of CTGF (P<0.01).

Unexpectedly, and in contrast to the cell data (FIG. 4), C3d deposition was not reduced by dabigatran. As shown previously, when examining C3a breakdown using a C3d antibody (group 2, clone 11) which recognizes the C3d epitope present in the C3a chain and its breakdown fragments, they can be distinguished according to their molecular weights (Thurman, J. M., et al. (2013). J Clin Invest 123, 2218-2230). At the end of the repair period (23 days), the C3α breakdown products C3d/C3dg and C3α′1 continue to be elevated with CNV (P<0.05), and both levels are further elevated by dabigatran (P<0.05) (FIG. 8B). Overall, the data show that in CNV, thrombin-mediated effects could be identified in the wound healing phase, but not during angiogenesis. Dabigatran was found to reduce CTGF and VEGF expression levels in CNV that was correlated with its effect on CNV lesion sizes. Surprisingly, and in opposition to the results identified in ARPE-19 cell cultures, inhibition of thrombin by dabigatran was found to increase the presence of complement breakdown products in RPE/choroid of CNV lesioned animals.

Complement inhibitors do not inhibit thrombin activity in a cell free system. It was investigated whether thrombin-mediated loss of barrier function is due to complement activation. To do so, it was confirmed that thrombin activity is not affected by complement inhibitors TT30 and compstatin in a cell-free system. Dabigatran or complement inhibitors were incubated with thrombin at a range of concentrations (10 mU/ml to 100 mU/ml), and the end-point thrombin activity was estimated from fluorescence intensity of the reaction mixture. Thrombin was shown to cleave its substrate in a concentration dependent manner as expected (FIG. 10). While dabigatran (10 μM) was found to significantly reduce the enzyme activity over the entire range of thrombin concentrations (10-500 U/mL), it was most effective between 10-100 U/mL (FIG. 10). In contrast, complement inhibitors TT30 (10 μM) and compstatin (100 μM) did not affect thrombin's enzymatic activity (FIG. 10B). No significant fluorescence signal reduction could be observed when using TT30 or compstatin, indicating these inhibitors do not interfere with thrombin activity.

C3α fragment generated by thrombin mediated C3 cleavage. Cleavage of C3α was evaluated using an antibody specific for C3a. As a control for molecular weight and specificity of 9-10 kDa band detected by the C3a antibody, samples were run in the presence of purified mouse C3a (Comptech). The 9-10 kDa band ran at the same molecular weight as purified C3a.

Discussion. The complement and coagulation system are two systems, in which activation leads to the assembly of proteolytic complexes, and their activities are tightly regulated by a set of specific activators and inhibitors. The proteolytic complexes are made up mainly of serine proteases that exhibit high substrate specificity. The crosstalk between these two systems has been studied in many diseases, with physiological consequences including the regulation of the immune system (Lupu, F., et al. Thromb Res 133 Suppl 1, S28-31; Kim, H. and E. M. Conway, Front Cardiovasc Med, 2019. Vol. 6: p. 131; and Fletcher-Sandersjoo, A., et al., Int J Mol Sci, 2020. 21(5)). The contributions of complement activation to pathology have been established in AMD, however, little is known about the coagulation system in this disease, or the interrelationship between coagulation factors and complement components. This study was focused on exploring the role of thrombin in AMD. The overall findings of this study are as follows: 1) dabigatran use reduces the risk for AMD, and in particular wet AMD in carefully matched Medicare populations, 2) thrombin induces its effects on RPE cell physiology via a dual mechanism, 3) thrombin cleaves complement component C3, C5 and activates the terminal complement pathway, leading to C3d and MAC deposition, 4) thrombin triggers both PAR-1 receptor and complement-mediated CTGF production, 5) thrombin induces VEGF secretion via both via PAR-1 receptor activation and complement activation, 6) dabigatran accelerates CNV lesion repair, and 7) modifying effects of dabigatran on CTGF and VEGF expression could be verified, but not of complement activation. Overall, a therapeutic effect of dabigatran was identified in patients, a mouse model of disease and a cell-based model. Crosstalk between thrombin and complement components was verified in the cell model (FIG. 8).

Thrombin is a zymogen, activated by coagulation factor X by proteolytic cleavages at Arg271 and Arg320 in a process of blood coagulation system activation (Adams, T. E. and J. A. Huntington, Biochimie, 2016. 122: p. 235-42). Association of dysregulated thrombin activation has been demonstrated in proliferative vitreoretinopathy (PVR), which is also a VEGF- and complement-associated (Grisanti, S., et al. (1991). Invest Ophthalmol Vis Sci 32, 2711-2717; and Ghasemi Falavarjani, K., and Modarres, M. (2014). Eye (Lond) 28, 1525-1526) ocular disease. In a late-stage, case-controlled study on AMD, levels of prothrombin fragment F1.2 (F1.2; a molecular marker of thrombin generation in vivo) was marginally lower in AMD compared with controls (Rudnicka, A. R., et al. (2010). Eye 24, 1199-1206). Dabigatran is a selective direct thrombin inhibitor and is, therefore, prescribed as an anticoagulant. It's mechanism of action is summarized in Dong et al., 2017, and includes reversibly binding to the active site of thrombin, preventing fibrinogen cleavage and thus the formation of insoluble fibrin. Dabigatran bound to thrombin prevents an important cleavage step of the extracellular N-terminal of the PAR1 receptor required for signaling (Bogatkevich, G. S., et al. (2009). Arthritis Rheum 60, 3455-3464), inhibiting many thrombin-induced profibrotic events, including collagen production by lung fibroblasts. Finally, thrombin activity has been shown to be involved in angiogenesis (Maragoudakis, M. E., et al. (2002). Biochem Soc Trans 30, 173-177), modulating VEGF secretion (Bian, Z. M., et al. (2007). Invest Ophthalmol Vis Sci 48, 2738-2746).

Using the MarketScan database, a large number of carefully matched patients were identified to examine whether reducing thrombin activity by dabigatran reduces the risk of AMD. The results described herein demonstrate that the risk of an AMD diagnosis and in particular wet AMD diagnosis is reduced in patients with prescriptions for dabigatran compared to a matched control group. Both the total rate of the new AMD cases and the rate of wet AMD events were reduced, independent of the years of exposure to the drug during the study period.

To understand mechanisms of thrombin-mediated changes in one of the cell types affected in AMD, ARPE-19 cells were grown as stable monolayers on transwell plates that have been used previously to analyze complement activation in response to oxidative stress (Thurman, J. M., et al. (2009). J Biol Chem 284, 16939-16947), smoke exposure (Kunchithapautham, K., et al. (2014). J Biol Chem 289, 14534-14546), as well as the complement pathway involved (Joseph, K., et al. (2013). J Biol Chem). The effects of thrombin activation was also examined as well as the cross-talk between thrombin and the complement system. RPE cells have been shown to express thrombin PAR1 and PAR3 receptors; however, the predominant form appears to be the PAR1 receptor (Yang, Z., et al. (1995). Invest Ophthalmol Vis Sci 36, 2254-2261; and Scholz, M., et al. (2004). Int J Mol Med 13, 327-331). The basal side of the RPE is exposed to blood-derived compounds and is expected to be protected, whereas the apical side is protected due to the barrier function of the RPE. However, upon destruction of the BRB in disease, both complement and thrombin can get access to the apical side. In initial experiments, the effects of thrombin when added to the basal or the apical side of the RPE was compared. TER measurements showed that thrombin caused reduction of TER when added to the apical side but not the basal side. In addition, no thrombin-mediated C3a cleavage products could be identified in the basal supernatant.

The cell-based study suggested that thrombin, via activation of PAR1 receptor signaling resulted in an increase in CTGF and VEGF expression. Interestingly, higher thrombin activity is present in vitreous fluid collected from PVR patient, and that PVR vitreous induces expression of cytokines/chemokines and growth factors in ARPE-19 cells (Bastiaans, J., et al. (2014). Invest Ophthalmol Vis Sci 55, 4659-4666), with many of these cytokines, chemokines, and growth factors facilitating the generation of a microenvironment for neovascularization in disease models (Heidemann, J., et al. (2003). J Biol Chem 278, 8508-8515; Huang, S. P., et al. (2004). J Biomed Sci 11, 517-527; Jo, N., et al. (2006). Am J Pathol 168, 2036-2053; Zheng, Q., et al. (2017) Tumour Biol; and Lou, Q., et al. (2020) Sheng Li Xue Bao 72, 441-448). In primary human RPE cells, dose- and time-dependent thrombin treatment was shown to induce VEGF-secretion via PAR1 receptor activation (Bian, Z. M., et al. (2007). Invest Ophthalmol Vis Sci 48, 2738-2746). One of the conclusions from this study is that thrombin does not trigger a unidirectional cell-signaling mechanism, but rather triggers multiple secondary messengers and works in combination with cytokines and growth factors (Bian, Z. M., et al. (2007). Invest Ophthalmol Vis Sci 48, 2738-2746). This might also explain why blocking complement or thrombin receptors alone did not completely block CTGF or VEGF expression and secretion. To reduce thrombin-mediated RPE pathology may therefore require a multipronged approach.

One of the major findings of the cell-based study is that thrombin can lead to the deposition of MAC on RPE cells as well as activate the complement cascade by cleaving complement component C3 and C5 into novel breakdown products, and therefore may be able to substitute for the requirement of C3/C5-convertase enzymes in RPE cells. In general, complement cascade activated by 3 pathways: the classical pathway (CP), the lectin pathway (LP), and the alternative pathway (AP). These pathways lead to activation of a series of serine proteases (C3 and C5 convertases), generate anaphylatoxins, opsonize the non-self/target cells, initiate MAC formation, and eventually lysed the cells (Muller-Eberhard, H. J. (1988) Annu Rev Biochem 57, 321-347; and Janeway Ca Jr, T. P., et al. (2001) Immunobiology: The Immune System in Health and Disease: The complement system and innate immunity. Garland Science, NY 5th edition.). In AMD, the AP appears to be the major contributor of complement activation leading to pathogenesis. The results described herein showed that thrombin activates the complement cascade in a non-conventional way, generating C3 and C5 breakdown products. The C3a breakdown products include a new 80 kDa fragment, a 32 kDa fragment and 9 kDa band that run at the same molecular weight as purified C3a. The C5a breakdown product included a new 35 kDa fragment that could be identified with a C5a antibody. The cleavage site was previously identified by Krisinger as the highly conserved thrombin-sensitive R947 site (Krisinger, M. J., et al. (2012). Blood 120, 1717-1725). Interestingly, in their study, they showed that the truncated C5b (C5b(T)) generated by thrombin in the context of a functioning complement system, was able to form a C5b(T)-9 MAC pore that was significantly more lytic than regular C5b-9 MAC (Krisinger, M. J., et al. (2012). Blood 120, 1717-1725). Support of the hypothesis that the 32 kDa C5a-containing fragment is bioactive comes from data that showed using ELISA that human C5 incubated with thrombin results in the generation of C5a or C5a-containing fragments, as the reaction products were able to induce chemotaxis in human neutrophils (Huber-Lang, M., et al. (2006). Nat Med 12, 682-687). However, thrombin activates complement signaling as well as inhibition by upregulating decay accelerating factor (DAF, CD45) expression on endothelial cells (Lidington, E. A., et al. (2000). Blood 96, 2784-2792). “Crosstalk” between components of the complement and the coagulation system has been summarized such that components of the coagulation cascade can cleave and/or activate proteins of the complement system in fluid phase, but proteins from the two cascades can interact on many different surfaces such as endothelial cell membranes (stationary surfaces) as well as circulating entities (Wiegner, R., et al. (2016). Immunobiology 221, 1073-1079). The data described herein on thrombin-mediated C3 cleavage (inhibited by compstatin, but not TT30) and MAC deposition (inhibited by TT30) as well as thrombin-mediated VEGF secretion (partially inhibited by compstatin and TT30), provide evidence for both mechanisms in ARPE-19 cells. In sum, these data show that thrombin can be useful to control aberrant complement activation.

In the mouse model of CNV, dabigatran was found to accelerate the resolution of the fibrotic scar. The fibrotic scar size over time was reduced by dabigatran, and was associated with a reduction in CTGF, and the amount of CTGF was highly correlated with the expression levels of VEGF (P<0.0001). Interestingly, in the long-term scar resolution model, the CNV-induced increase in complement activation was not reduced, but rather significantly increased for the C3α breakdown products analyzed. The CNV model supports the findings in patients, that wet AMD risk is reduced by dabigatran use. The molecular analysis in the mouse model supports the findings in ARPE-19 cell monolayers that dabigatran reduces thrombin-induced CFGF expression and thereby reduces the amount of VEGF.

In the eye, complement homeostasis is influenced by both levels of systemic circulating complements and ocular complement production (Clark, S. J., and Bishop, P. N. (2018). Seminars in Immunopathology 40, 65-74). To regulate an overactive AP pathway in the RPE/BrM/choriocapillaris, the main targets of complement activation in AMD impose challenges. Using systemic delivery of complement inhibitors may be limited by BrM, which restricts access for larger and glycosylated proteins (Clark, S. J., et al. (2017). Front Immunol 8, 1778), whereas intravitreal administration of drug in the context of a healthy or partially damaged BRB may show limited transport of therapeutics across the RPE due to a lack of appropriate transport systems (Strauss, 0. (2005). Physiol Rev 85, 845-881). Additional challenges were also observed for anti-VEGF treatments, which are currently standard of care for patients with wet AMD. It has been shown that anti-VEGF responses are recipient-dependent, and therefore complicate dose optimization (Amoaku, W. M., et al. (2015). Eye (Lond) 29, 721-731; and Schargus, M., and Frings, A. (2020). Clin Ophthalmol 14, 897-904). Many patients convert from intermediate, presumably complement dependent AMD, to wet AMD, requiring anti-VEGF treatment, followed by conversion to geographic atrophy. To target each of the regulators (complement/VEGF) individually might complicate AMD treatment options further. Hence, the identification of a compound that acts upstream of both of these two pathways such as thrombin, can be a useful new treatment. Dabigatran etexilate is an approved oral drug that is capable of lowering thrombin activity in systemic circulation and specific organs such as lung, heart and brain (Bogatkevich, G. S., et al. (2011). Arthritis Rheum 63, 1416-1425; and Devereaux, P. J., et al. (2018). Lancet 391, 2325-2334), and can lower thrombin-mediated outcomes in AMD.

1. A method of treating age-related macular degeneration in a subject, the method comprising: administering to the subject in need thereof a therapeutically effective amount of a direct thrombin-inhibitor.

2. The method of claim 1, wherein the direct thrombin-inhibitor is dabigatran.

3. The method of claim 1, the subject has been diagnosed with age-related macular degeneration prior to the administering step.

4. (canceled)

5. (canceled)

6. The method of claim 2, wherein the dabigatran is administered orally, parenterally, by intravitreal injection, eye drop, or depot.

7. (canceled)

8. (canceled)

9. The method of claim 2, wherein the therapeutically effective amount of dabigatran is 20 mg to 300 mg/day.

10. The method of claim 1, further comprising administering a therapeutically effective amount of an anti-VEGF therapeutic, a complement therapeutic or an antioxidant.

11. The method of claim 2, wherein the administration of dabigatran reduces or ameliorates one or more symptoms of age-related macular degeneration.

12. The method of claim 11, wherein the one or more symptoms of age-related macular degeneration are blurry or fuzzy vision; difficulty recognizing familiar faces; straight lines appear wavy; a dark, empty area or blind spot in the center of vision; loss of central vision; and the presence of drusen.

13. The method of claim 2, wherein the administration of dabigatran reduces complement activation, C3d deposits, membrane attack complex of complement (MAC) deposits or a combination thereof in the subject.

14. The method of claim 2, wherein the administration of dabigatran decreases expression of connective tissue growth factor in the subject.

15. A method of inhibiting expression of connective tissue growth factor in a subject, the method comprising: administering to the subject in need thereof a therapeutically effective amount of dabigatran.

16. (canceled)

17. A method of reducing complement activation, C3d deposits and/or membrane attack complex of complement (MAC) deposits in a cell, a tissue, a membrane or an extracellular matrix, the method comprising: contacting a cell or tissue or administering to a subject in need thereof, a therapeutically effective amount of a dabigatran.

18.-21. (canceled)

22. The method of claim 15, wherein the dabigatran is administered orally, parenterally parenterally, by intravitreal injection, eye drop, or depot.

23. (canceled)

24. The method of claim 17, wherein the dabigatran is administered orally, parenterally, by intravitreal injection, eye drop, or depot.

25. The method of claim 15, wherein the therapeutically effective amount of dabigatran is 20 mg to 300 mg/day.

26. The method of claim 15, further comprising administering a therapeutically effective amount of an anti-VEGF therapeutic, a complement therapeutic or an antioxidant.

27. (canceled)

28. The method of claim 15, wherein complement activation is decreased.

29. The method of claim 17, wherein the therapeutically effective amount of dabigatran is 20 mg to 300 mg/day.

30. The method of claim 17, further comprising administering a therapeutically effective amount of an anti-VEGF therapeutic, a complement therapeutic or an antioxidant.
